(19) United States
(12) Patent Application Publication
(10) Pub. No.: US 2006/0191023 A1
(43) Pub. Date: Aug. 24, 2006

(54) Automated method for high throughput screening of nematodes

(76) Inventors: Matthew S Gill, Novato, CA (US); Anders Olsen, Novato, CA (US); Gordon J. Lithgow, Novato, CA (US)

Correspondence Address: QUINE INTELLECTUAL PROPERTY LAW GROUP, P.C., P O BOX 458, ALAMEDA, CA, 94501, US

(21) Appl. No.: 10/530,091

(22) PCT Filed: Oct. 9, 2003
(86) PCT No.: PCT/US03/32066

§ 371 Date: Mar. 13, 2006

(30) Foreign Application Priority Data
Oct. 9, 2002 (US) 60417465

(51) Int. Cl. A01K 67/033

(52) U.S. Cl. 800/3; 800/13

(57) ABSTRACT

This invention provides a high throughput survival assay, using uptake of a marker dye (e.g. a fluorescent dye) as a marker of death of a nematode. The assay permits high throughput screening of thousands of compounds possible. By the application of automated worm handling technology we are able to accurately dispense nematodes into 384 well microtitre plates, at rates many thousand of Limes faster than previously possible. In addition, we have automated the analysis of survival by the use of a fluorometric plate reader that quantitates the degree of fluorescence within each well.

### CROSS-REFERENCE TO RELATED APPLICATIONS

This application claims benefit of and priority to U.S. Ser. No. 60/417,465, filed on Oct. 9, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the field of high-throughput screening. In particular, this invention provides materials and methods for high-throughput screening of nematodes, e.g. for susceptibility to one or more test agents.

### BACKGROUND OF THE INVENTION

Many genetic or environmental manipulations that extend lifespan also enhance survival following acute stress. For example, young adult C. elegans worms carrying mutations in insulin/IGF-1 signaling genes that extend lifespan also exhibit resistance to a range of stresses including heat (Lithgow et al. (1995)Proc. Natl. Acad. Sci., USA, 92: 7540-7544), oxygen radicals (Larsen (1993Proc. Natl. Acad. Sci., USA, 90: 8905-8909) and heavy metal toxicity (Barsyte et al. (2001) FASEB J 15: 627-634). Such observations are echoed in other species where longevity and stress resistance are correlated. Furthermore, isolation of long-lived mutants can be greatly expedited by selecting for their elevated stress resistance phenotype rather than performing lengthy lifespan analyses (Sampayo et al. (2000) Ann N Y Acad Sci 908:324-326). This strategy of using a longevity surrogate can also be applied to the identification of drugs which confer stress resistance and therefore lifespan extension.

Despite the advances in the biology of aging in C. elegans, high throughput screens have remained impractical, due to severe limitations on the number of experiments that can be conducted manually. In lifespan analyses, touch provoked movement remains the best score of survival and requires microscopic examination of each individual worm. This method works well in the hands of experienced investigators yet the score remains subjective and can be difficult to perform on old and fragile animals.

### SUMMARY OF THE INVENTION

In order to develop a rapid and objective score of survival we have used the fluorescent dye SYTOX®, developed by Molecular Probes Inc (OR), which is a nucleic acid stain that binds to DNA in cells whose membrane has been compromised. This compound does not in itself affect the viability of the nematodes and upon binding to DNA, fluorescence can be quantified using a fluorometer.

A prerequisite for high throughput screening is the preparation of large numbers of microtitre plates containing nematodes. We have employed COPAS™ BIOSORT™ automated worm handling technology to dispense individual nematodes into 384 well plates containing SYTOX®, following which fluorescence is quantified in a fluorometric plate reader. We have validated this technique by assessing survival in response to an acute heat stress and we find that we can reproducibly identify a thermotolerance difference between the wild type strain of C. elegans N2 and the mutant strain age-1, TJ1052(hx546), which is known to be thermot olerant and long lived (1). In addition we can detect an increase in thermotolerance in wild type worms treated with a compound that pharmacologically mimics the age-1 mutation (Babar et al. (1999) Aging 20: 513-519).

By combining automated plate preparation with fluorometric quantitation we have developed a technique that allows for a rapid and objective score of survival in individual worms at rates many times faster than previously possible. This system provides an opportunity to perform high throughput screens to investigate many thousands of compounds for their effect on any fluorescent signal of interest.

Definitions

A “detectable label that indicates the viability of a cell or organism” is a label that differentially labels a cell or organism depending on the viability of the cell or organism. Thus such a detectable label will differentiate cells or organisms that are dead or dying from cells or organisms that are alive and healthy.

The term microtiter plate refers to an apparatus comprising a plurality of wells within which can be disposed reagents, and/or cells, and/or nematodes or other organisms, and the like. Commercially available microtiter plates are typically commercially available in 96 well, 100 well, 320 well, a 384 well, 864 well, and 1536 well formats. The microtiter plates can be clear or opaque and can be fabricated out of low fluorescence materials.

“SYTOX” (available from Molecular Probes, Inc.) is a green nucleic acid stain that easily penetrates compromised cell membranes, but is completely excluded from live eukaryotic and bacterial cells. After a brief incubation with SYTOX Green stain, dead cells fluoresce bright green when excited with any 470 to 490 nm source. Combined with its >500-fold fluorescence enhancement upon nucleic acid binding, these properties make SYTOX Green stain a simple and quantitative single-step indicator for dead eukaryotic and bacterial cells.

A “genetic knock out” refers to an organism in which the normal activity/expression of one or more genes is disrupted/inhibited.

A “test agent” refers to refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term small organic molecules refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The terms “label” or “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The term “transgenic nematodes” refers to nematodes recombinantly modified or derived from recombinantly modified nematodes to contain a gene not typically found in the nematodes and/or that to contain a gene in a number of copies not typically found in nematodes, and/or to express a gene at a level typically not found in a nematode.

### BRIEF DESCRIPTION OF THE DRAWINGS

FIGS. 1A-1D illustrate the evaluation of plate formats for detecting fluorescence from 4-day old nematodes stained with the nucleic acid stain SYTOX®. Fluorescence was measured in a Fluoroskan Ascent fluorometer with a 485 nm excitation filter and 538 nm emission filter. FIG. 1A: Schematic diagram illustrating the principle by which fluorescence is quantified from the wells of a microtitre plate in the Fluoroskan Ascent fluorometer (Thermo Labsystems, Finland). Light passes from its source above the microtitre plate, through a filter which generates the excitation wavelength into the well. The fluorescence generated within the wall is reflected by the semitransparent mirror through an angle of 90°, where it then passes through the emission filter onto the detector. FIG. 1B: Panel (i) Measurement of SYTOX®-associated fluorescence in live and dead worms dispensed into wells of a transparent polystyrene 96 well plate. Panel (ii) Model for the optical properties of the transparent plate. When the excitation beam reaches the well some is lost by transmission through the plate and some is reflected back to the detector, resulting in high baseline fluorescence. Similarly some of the light emitted by a fluorescent object will be lost by transmission. Thus the detectable fluorescence will be reduced. These factors combine to generate a small difference in fluorescence between live and dead worms stained with SYTOX®. FIG. 1C: Panel (i) Measurement of SYTOX®-associated fluorescence in live and dead worms dispensed into wells of an opaque white polystyrene 96 well plate. Panel (ii) Model for the optical properties of the white plate. The entire excitation beam is reflected back to the detector, resulting in very high baseline fluorescence. As a result the emission by a fluorescent object will be almost completely masked by this reflected signal. FIG. 1D: Panel (i) Measurement of SYTOX®-associated fluorescence in live and dead worms dispensed into wells of an opaque black polystyrene 96 well plate. Panel (ii) Model for the optical properties of the black plate. The entire excitation beam is mostly absorbed by the surface of the plate generating a low baseline level. When a fluorescent object excited the emitted fluorescence is detected with relatively little loss of signal. This results in a good separation between live, non-fluorescing and dead, fluorescing worms.

FIGS. 2A and 2B show a comparison of plate formats for detecting fluorescence from 4-day old nematodes stained with the nucleic acid stain SYTOX. Fluorescence was measured in a Fluoroskan Ascent fluorometer with a 485 nm excitation filter and 538 nm emission filter. FIG. 2A: Panel (i) Measurement of SYTOX®-associated fluorescence in an opaque black 96 well plate. The fluorescence in some wells containing a dead worm was similar to that found in live non-fluorescing worms despite the presence of a highly fluorescent object. Panel (ii) The diameter of the excitation beam is 3 mm while the diameter of the well is 3 mm. Thus a worm which is lies outside or partly overlaps with the excitation beam will generate a lower fluorescent signal. FIG. 2B: Panel (i) Measurement of SYTOX®-associated fluorescence in an opaque black 384 well plate. There was complete separation between live and dead worms in terms of fluorescence. Panel (ii) The diameter of the excitation bean is 3 mm and the diameter of the well is 3 mm. Thus fluorescence can be detected irrespective of the position of the worm in the well.

FIGS. 3A and 3B illustrate the assessment of thermotolerance using a fluorometer to quantify the SYTOX®-associated fluorescence from individual 4-day old nematodes dispensed into the wells of a 384 well black microtitre plate. FIG. 3A: Panels (i) and (ii) Fluorescence as a function of time for individual nematodes heat shocked at 35° C. The wild type strain N2 is represented by the black lines and the mutant strain TJ1052[age-1(hx546)] is denoted by the red lines. FIG. 3B: Kaplan-Meier survival curve illustrating mean fraction surviving (±SEM) for N2 (black) and TJ1052[age-1(hx546)] derived from the individual fluorescence curves (p=0.0002). The wild type strain N2 is represented by the black circles and the mutant strain TJ1052[age-1(hx546)] is denoted by the red circles.

FIG. 4 shows the effect of LY294002 on thermotolerance of 4-day old nematodes. Kaplan-Meier survival curve illustrating mean fraction surviving (±SEM) for N2 (black circles) and N2 treated with LY294002 at a concentration of 100 nM (red circles) or 10 μM (green triangles) compared to TJ1052[age-1(hx546)] (yellow triangles).

FIG. 5, panels A through E illustrate assessment of thermotolerance using SYTOX green. 3 day old gravid hermaphrodites, grown in liquid culture at 25° C., were subjected to a lethal heat shock at 35° C. Bright field (Panel A) and fluorescent view (Panel B) of a live, adult wild-type (N2) nematode in 1 μM SYTOX. Bright field view (Panel C) and fluorescent view (Panel D) of a heat-killed adult nematode in 1 μM SYTOX. (Panel E) Kaplan-Meier survival curve illustrating the fraction of animals surviving (±SEM) for wild-type N2 (n=49) and TJ1052[age-1(hx546)] (n=49). TJ1052[age-1(ILx546)] was significantly more thermotolerant than the wild-type strain (p<0.0001, Log-rank test).

FIGS. 6A and 6B illustrate the assessment of survival in response to oxidative stress. 3 day old gravid hermaphrodites, grown in liquid culture at 25° C., were treated with 40 mM paraquat for 48 hours and then scored for survival by microscopy. (FIG. 6A) Survival of wild-type worms scored for touch provoked movement (manual score) and then for fluorescence by microscopy 15 minutes later (SYTOX score). (FIG. 6B) Survival of wild-type worms grown in the presence of 0.5 mM Euk-134, scored for touch provoked movement (manual score) and then for fluorescence by microscopy 15 minutes later (SYTOX score).

FIG. 7, panels A through H illustrate the automated assessment of thermotolerance. 6 day old gravid hermaphrodites grown at 15° C. were subjected to lethal heat shock at 37° C. using the automated thermotolerance assay. (Panel A) Individual fluorescence curves for 4 representative adult hermaphrodite C. elegans heat shocked at 37° C. in S-medium+1 μM SYTOX in the absence of bacteria (solid line TJ1060; dashed line TJ1062[age-1(hx542)]; dot-dashed line indicates fluorescence threshold used to score time of death). (Panel B) Kaplan-Meier curve illustrating fraction alive (±SEM) for TJ1060 (●) and TJ1062[age-1(hx542)] (∘) in S-medium+1 μM SYTOX (p=n.s., Log-Rank test). (Panel C) Individual fluorescence curves for 4 adult hermaphrodite C. elegans heat shocked at 37° C. in S-medium+10 cells/ml E. coli OP50+1 μM SYTOX (solid line TJ1060; dashed line TJ1062[age-1(hx542)]; dot-dashed line indicates fluorescence threshold used to score time of death). (Panel D) Kaplan-Meier curve illustrating fraction alive (±SEM) for TJ1060 (●) and TJ1062[age-1(hx542)] (∘) in S-medium+10⁷ cells/ml E. coli OP50+1 μM SYTOX (p<0.0001, Log-Rank test). (Panel E) Individual fluorescence curves for 4 adult hermaphrodite C. elegans heat shocked at 37° C. in S-medium+10⁸ cells/ml E. coli OP50+1 μM SYTOX (solid line TJ1060; dashed line TJ1062[age-1(hx542)]; dot-dashed line indicates fluorescence threshold used to score time of death). (Panel F) Kaplan-Meier curve illustrating fraction alive (±SEM) for TJ1060 (●) and TJ1062[age-1(hx542)] (∘) in S-medium+10⁸ cells/ml E. coli OP50+1 μM SYTOX (p<0.0001, Log-Rank test). (Panel G) Individual fluorescence curves for 4 adult hermaphrodite C. elegans heat shocked at 37° C. in S-medium+2×10⁸ cells/ml E. coli OP50+1 μM SYTOX (solid line TJ1060; dashed line TJ1062[age-1(hx542)]; dot-dashed line indicates fluorescence threshold used to score time of death). (Panel H) Kaplan-Meier curve illustrating fraction alive (±SEM) for TJ1060 (●) and TJ1062[age-1(hx542)] (∘) in S-medium+2×10⁸ cells/ml E. coli OP50+1 μM SYTOX (p<0.0001, Log-Rank test).

### DETAILED DESCRIPTION

This invention pertains to the development of a high-throughput screening system and methods of use thereof to evaluate the effect of one or more test agents on a nematode.

The assays comprise a microtitre plate based survival assay, using uptake of a detectable label that indicates nematode viability (e.g. a fluorescent dye as a marker of death), which makes high throughput screening of thousands of compounds possible. By the application of automated worm handling technology we are able to accurately dispense nematodes into 384 well (or higher well number) microtitre plates, at rates many thousand of times faster than previously possible. In addition, in certain embodiments, we have automated the analysis of survival by the use of a fluorometric plate reader that quantitates the degree of fluorescence within each well.

The microtitre plate assay we describe offers a considerable increase in throughput and a reduction in investigator time compared with the conventional survival assay in which touch provoked movement is assessed. By utilising robotic technology, throughput can be increased by orders of magnitude, making it possible to screen thousands of compounds at a time. Moreover the application of this technique is not only limited to screening for drugs or other agents that affect survival in nematodes but it can also be applied any to situation where the is a viability measure.

Thus, in one embodiment, this invention provides a method for screening for the activity of one or more agents on a nematode (or other organism) and/or for the susceptibility of a nematode (or other organism) to said agents. The method involves providing a microtiter plate comprising a plurality of wells where each of a plurality of the wells contains one or more nematodes. The nematodes are contacted with one or more test agents and with a detectable label that indicates the viability of the nematodes. The label is then detected and/or quantified to ascertain the viability of nematodes contacted with the test agent(s). An increase or decrease in the viability of the nematodes contacted with the test agents(s) indicates that the nematodes are susceptible to the activity of the test agent(s) and/or that the test agent(s) are active on the nematodes.

The increase or decrease in viability can be as compared to an appropriate positive and/or negative control. A typical negative control will be the same assay run with the test agent(s) at a lower concentration or absent. A typical positive control will be the same assay run with the test agent(s) at a higher concentration or absent.

In preferred embodiments, the increase or decrease will be a detectable increase or decrease. In more preferred embodiments, the increase or decrease is a statistically significant increase or decrease (e.g. at the 90%, 95%, or 99% confidence level, as compared to the control or controls) e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change. Preferred reductions include at least a 5% reduction, more preferably at least a 10% reduction, and most preferably at least a 15% or 20% reduction in neurological damage.

While a wide variety of detectable labels are useful for the assays described herein, in certain embodiments, the detectable label is a label that penetrates compromised cell membranes, but is substantially or completely excluded from live eukaryotic cell (e.g., SYTOX® fluorescent labels).

In certain embodiments, this invention provides devices and/or systems for running the assays described herein. One such device comprises a microtiter plate comprising a plurality of wells, each of a plurality of the wells containing one or more nematodes and/or a detectable label as described herein.

Integrated systems can additionally comprise one or more automated devices for loading the microtiter plates with nematodes and/or detectable label(s), and/or test agent(s), and/or automated devices for reading the results of the assay (e.g. fluorometers). Such integrated systems can optionally include additional robotics for sample processing, reagent synthesis, microtiter plate storage and/or incubation and/or handling, computer systems for controlling the devices and recording and/or analyzing assay data and the like.

High Throughput Screening

As indicated above, the assays of this invention are also amenable to “high-throughput” modalities. Conventionally, new chemical entities with useful properties (e.g., increasing thermotolerance or resistance to oxidative stress) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

Combinatorial Chemical Libraries for Test Agents.

The likelihood of an assay identifying a desired test agent (e.g. an agent that increases thermotolerance or resistance to oxidative stress) is increased when the number and types of test agents used in the screening system is increased. Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

High Throughput Assays of Chemical Libraries.

The assays described herein are particularly well suited to high throuput modalities. “The assays can be run in standard commercially available microtiter plates (e.g. 96 well, 100 well, 320 well, 384 well, 864 well, and 1536 well formats). A single test agent can be screened in a single well on the microtiter plate, however, in certain embodiments, multiple test agents can be screened for activity in a single well. Those wells that test positive can then be deconvolved in subsequent assays to determine which of the test agents in the positive screen was responsible for the positive signal.

Thus, for example, where 10 agents are tested in each well, a single 384 well microtiter plate can be used to screen 3,840 different test agents, and only 10 plates are required to screen for 38,400 test agents.

The assays are thus suited for massive parallelism and literally thousands of test agents can be screened in a single day.

Kits.

In another embodiment, this invention provides kits for practice of the methods of this invention. In certain embodiments, such kits typically comprise a microtiter plate comprising a plurality of wells; a plurality of nematodes; and a detectable label that indicates the viability of the nematodes. In certain embodiments, the nematodes can be disposed such that each of a plurality of the wells contains one or more of said nematodes.

In certain embodiments, a plurality of the wells of the microtiter plate also contain the detectable label, and in certain embodiments, a plurality of the wells containing nematodes also contain the detectable label.

A variety of detectable labels, as described herein, can be included in the kit. In certain embodiments, the detectable label is a label that penetrates compromised cell membranes, but is substantially or completely excluded from live eukaryotic cells. Certain preferred labels include, but are not lmited to SYTOX® blue, SYTOX® orange, and propidium iodide.

The nematodes can include, but are not limited to wild-type nematodes, inbred strains, recombinantly modified nematodes including, but not limited to knockouts and/or transgenic nematodes.

The kit can optionally also include instructional materials containing directions teaching the use of one or more components of the kit in high throughput screening assays, e.g. as described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Development of an Automated Method for the Quantification of Fluorescence in the Nematode Caenorhabditis Elegans and its Application to High Throughput Drug Screening

Summary

Many genetic or environmental manipulations that extend lifespan in the nematode Caenorhabditis elegans (C. elegans) also enhance survival following acute stress. Using stress resistance as a surrogate measure of lifespan in genetic screens it is has been possible to identify new genes that affect the aging process much more efficiently. Similarly this strategy can be applied to screening of pharmacological agents that extend lifespan.

We have developed a microtitre plate based survival assay, using uptake of a fluorescent dye as a marker of death, which makes high throughput screening of thousands of compounds possible. By the application of automated worm handling technology we are able to accurately dispense nematodes into 384 well microtitre plates, at rates many thousand of times faster than previously possible. In addition, we have automated the analysis of survival by the use of a fluorometric plate reader that quantitates the degree of fluorescence within each well.

The microtitre plate assay we describe offers a considerable increase in throughput and a reduction in investigator time compared with the conventional survival assay in which touch provoked movement is assessed. However, by utilising robotic technology, throughput could be increased by orders of magnitude, making it possible to screen thousands of compounds at a time. Moreover the application of this technique is not only limited to screening for drugs that affect survival in nematodes but it could also be applied to situation where the endpoint is a fluorescent signal.

Methods

Materials

2-(4-Morpholinyl)-8-phenyl-4H-1-benzopyran-4-one (LY-294002; Sigma-Aldrich, St Louis, Mo.).

SYTOX® green nuclei acid stain (Molecular Probes, Portland, Oreg.).

Nematode Culture

The Bristol N2 (wild-type) strain and TJ1052[age-1(hx546)II] were obtained from the Caenorhabditis Genetic Center at the University of Minnesota. Worms were maintained on 10 cm nematode growth medium (NGM) agar plates carrying a lawn of OP50, a leaky uracil-requiring strain of Escherichia coli (Wood (1988) Introduction to C. elegans biology. In: Wood WB (ed). The nematode Caenorhabditis elegans. Cold Spring Harbour Laboratory Press, Cold Spring Harbour: 1-16). E. coli strain OP50 was obtained from the Caenorhabditis Genetic Center. Liquid culture was undertaken in S-medium with a minimum concentration of 10⁹ cells/ml E. coli OP50 (Id.). Culture plates were maintained at 20° C., unless otherwise stated. Eggs were prepared hypochlorite treatment of gravid adults (Id.). For live versus dead comparisons, nematodes were heat-killed at 60° C. in a water bath.

Dispensing Worms into Microtitre Plate

Worms (˜10,000 to 20,000) were washed off NGM plates with 10 ml S-basal into a 15 ml tube. After gravity settling for 5 minutes, 9 ml S-basal was aspirated and replaced with fresh 9 ml S-basal. Following a further 2 washes the nematodes were introduced to the sample cup on the COPAS™ BIOSORT at a concentration of 1 worm per μl. Worms were dispensed, 1 animal per well, into either 96 well or 384 well microtitre plates according to the manufacturers instructions. For thermotolerance assays 384 well low volume plates (Greiner Bio-One Inc, Longwood, Fla.) were prepared with 20 μl S-medium containing SYTOX® at a final concentration of 1 μM. After the nematodes were dispensed the plate was sealed using VIEWseal™ plate sealing film (Greiner Bio-One Inc, Longwood, Fla.) to prevent evaporation.

Microplate Thermotolerance Assay.

The thermotolerance assay was performed in a Fluoroskan Ascent fluorometer (Thermo Labsystems, Finland). The ambient temperature of the fluorometer was set to 35° C. and Ascent software was configured to measure the fluorescence from each well every 30 minutes over a 20-24 hour period with a 20 millisecond measurement time for each well. At this setting a complete 384 well plate could be read within 30 seconds. The excitation wavelength for fluorescence was 485 nm and the emission wavelength 538 nm.

Statistical Analysis.

Analysis of individual fluorescence curves was performed using the Fluoroskan Ascent software. A 100% increase in fluorescence over baseline was used a cut-off to define a death event. Differences in thermotolerance were assessed using the Mantel-Haenszel logrank test as implemented in Prism® (GraphPad Software Inc.,). Kaplan-Meier survival curves were generated by using Prism® survival analysis.

Results

Validation of Microtitre Plate Format

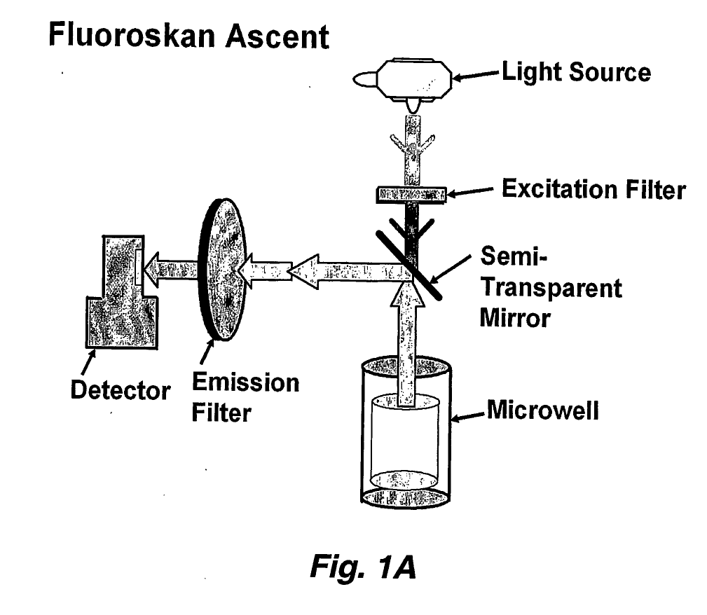

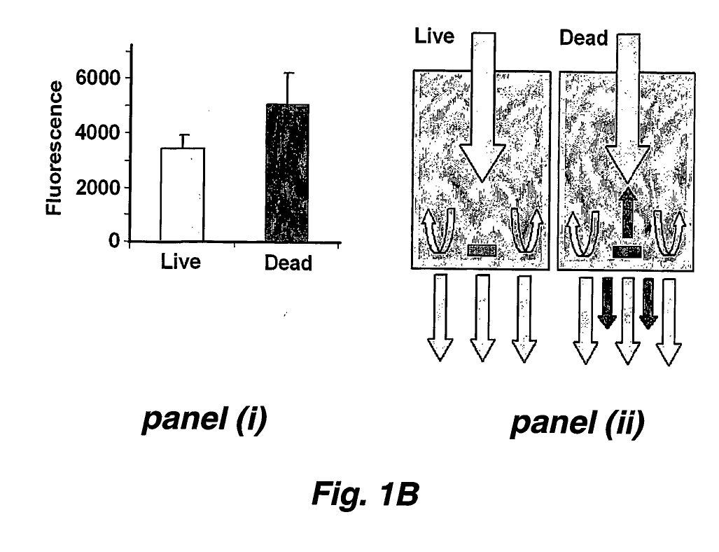

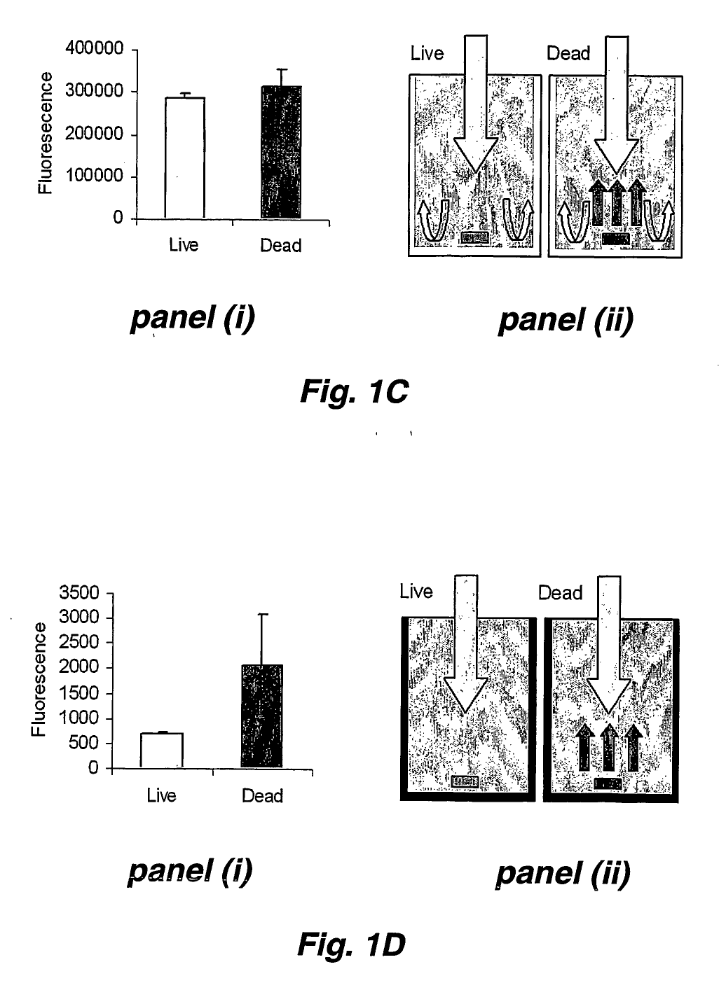

In the Fluoroskan fluorometer the light source which provides the excitation beam is positioned above the well and the resulting emission is reflected perpendicularly to the reach the detector (FIG. 1A). The optical characteristics of the microtitre plates were investigated in order to provide the greatest separation between the autofluorescence of live animals and the SYTOX®-associated fluorescence from dead animals. Single live worms and single dead worms that had been stained with SYTOX® were dispensed into individual wells of 3 different types of 96 well microtitre plates: transparent polystyrene, white opaque polystyrene and black opaque polystyrene. The fluorescence within each well was measured within the Fluoroskan and the difference in fluorescence between live and dead worms assessed in each plate type (FIG. 1B-D). The black opaque plates generated the greatest degree of separation between live, non-fluorescing worms and dead, fluorescent animals (FIG. 1D).

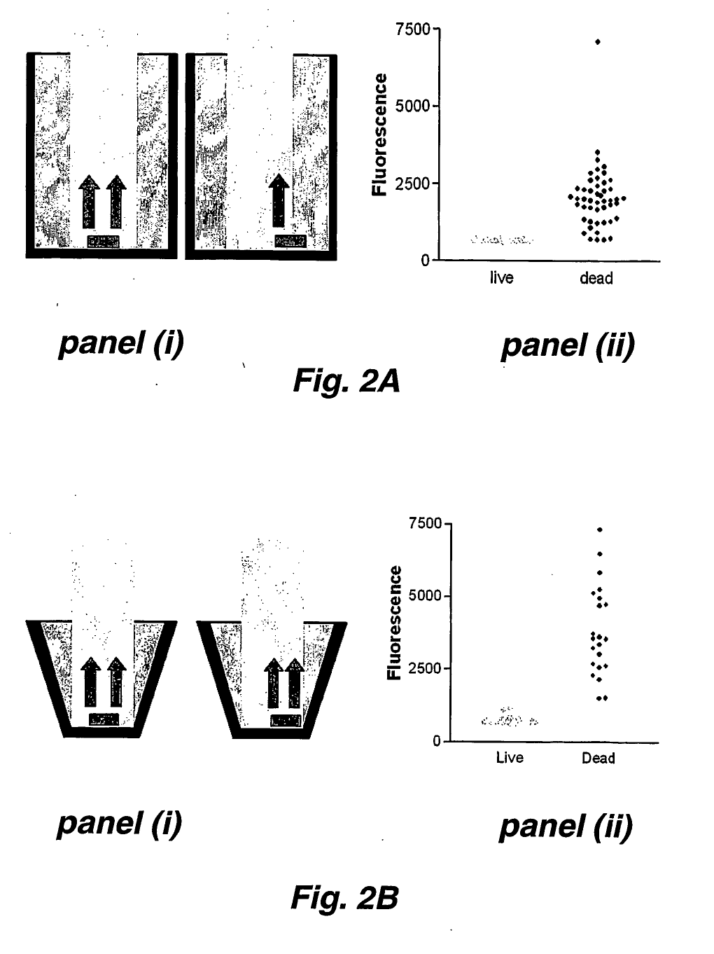

It was apparent that some wells in the 96 well plate gave a baseline level of fluorescence despite there being a highly fluorescent object within the well. The diameter of the well in the 96-well plate was 6 mm whilst the diameter of the excitation beam was only 3 mm. Thus a worm located on the perimeter of the well would not be detected (FIG. 2A). To circumvent this problem we used 384 well opaque black plates, which have a well diameter of 3 mm and consequently all fluorescing worms could be detected (FIG. 2B).

Thermotolerance Assay

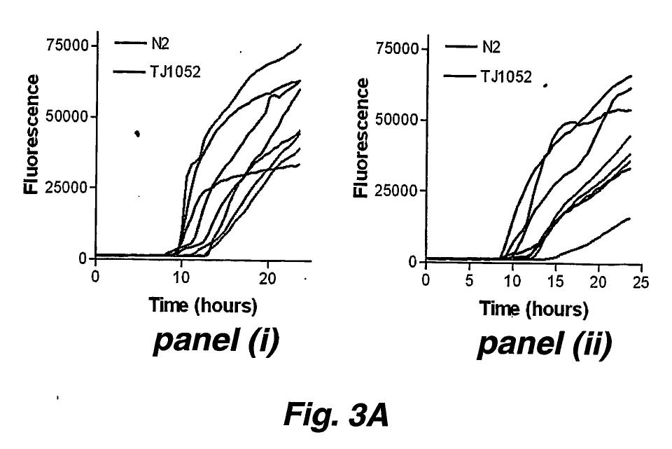

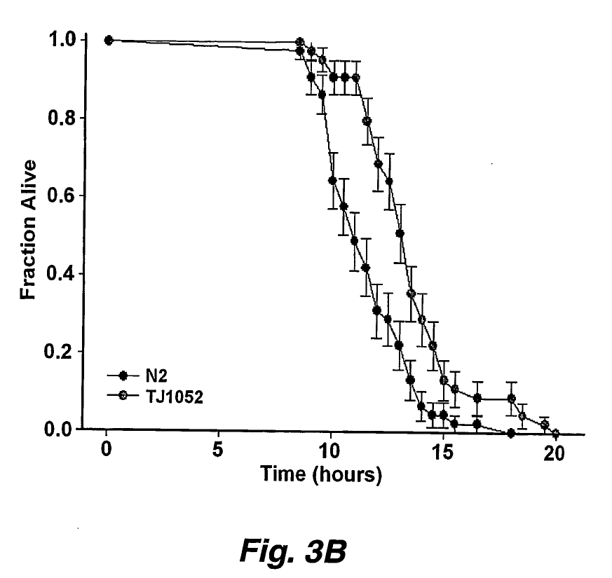

It has previously been demonstrated that worms which carry the age-1(hx546) mutation exhibit significant thermotolerance compared to the wild type strain N2, when incubated at 35° C. In order to validate the microplate heat shock assay we dispensed 192 wild type worms and 192 TJ1052 into individual wells of a 384 well plate, containing 20 μl S-medium+1 μM SYTOX®. The plate was incubated at 35° C. in the fluorometer and fluorescence measured every 30 minutes for 24 hours. FIG. 3A shows the individual fluorescence curves for a sample of animals. The baseline fluorescence reading from live animals was approximately 1000 fluorescence units and death was associated with a sharp increase in fluorescence. It can be seen from FIG. 3A that the curves associated with TJ1052 are generally to the right of those of N2, indicating that the onset of fluorescence, and thus death, was delayed in TJ1052. We defined the time of death as the time at which the fluorescence exceeded the baseline by 100%, and plotted the data as a Kaplan-Meier survival curve (FIG. 3B). The worms carrying the age-1(hx546) mutation were significantly more thermotolerant compared to wild type (p=0.0002).

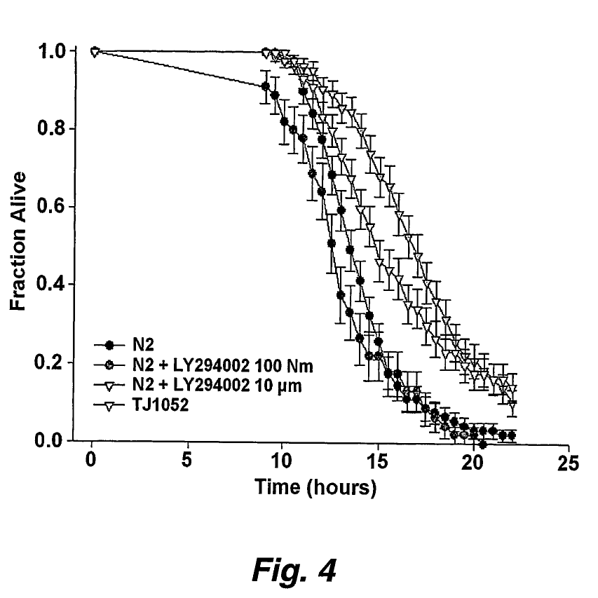

In order to demonstrate that the microplate assay could be used to detect a pharmacologically-induced increase in thermotolerance we used the compound LY-294002 (2-(4-Morpholinyl)-8-phenyl-4H-1-benzopyran-4-one). This compound is a specific inhibitor of phosphatidylinositol 3-kinase (PI3K), the molecule thought to be altered in age-1(hx546). It has been previously shown that treatment of wild type worms with LY294002 confers thermotolerance similar to that seen in age-1(hx546) (Babar et al. (1999) Aging 20: 513-519). We grew wild-type animals from eggs to 4 day adults in S-medium containing either 10 μM or 10 nM LY294002 at 20° C. and compared them to untreated wild type and age-1(hJ546). Both age-1(hx546) and 10 μM LY294002 treated worms were significantly more thermotolerant than untreated wild type worms (both p<0.0001, FIG. 4). There was no significant effect of the drug at 100 nM (p=0.1257).

Discussion

Considerable advances in our understanding of the aging process have come from studies in model systems, such as the nematode roundworm C. elegans (Lithgow (1996) Bioessays 18: 809-815). These genetic studies have suggested that the factors which control the rate of aging are conserved between such evolutionarily distant species as nematodes, flies and mammals. The challenge is now to “solve” the biology of aging in these systems and quickly apply the emergent knowledge to human aging and age-related disease. In this respect, a range of pharmacological compounds with anti-aging properties would be of enormous value in facilitating the study normal mammalian aging, functional decline and age-related disease. The assay we describe for assessing survival of individual nematodes in a microtitre plate format now makes this type of high throughput screen a realistic proposition.

We have previously demonstrated that C. elegans is a useful bioassay for assessing compounds that affect lifespan. We found that treatment of nematodes with synthetic catalytic mimetics (SCMs) that exhibit superoxide dismutase and catalase activities generated a significant increase in lifespan (Melov et al. (2000) Science 289: 1567-1569). However, whilst our discovery, and others (Kang et al. (2002)Proc. Natl. Acad. Sci., USA, 99: 838-843), has demonstrated that pharmacological intervention in aging is practicable, these experiments are extremely labor intensive. Thus, the approach has been to target those compounds that are likely to impact on known aging pathways rather than to screen thousands of compounds whose mechanism of action and molecular targets are not known.

The development of this high throughput assay system was achieved by the application of three separate, existing technologies: COPAS™ BIOSORT automated nematode sorting and dispensing platform (Union Biometrica, MA); SYTOX® fluorescent dye (Molecular Probes, OR); and Fluorsokan Ascent fluorometer (Thermo Labsystems, Finland).

The COPAS™ BIOSORT “wormsorter” system was used to dispense a single worm into the wells of a 384 well plate, a task it can perform in less than 1 minute depending on the heterogeneity of the source population. It would take considerably longer for an investigator to manually dispense this number of worms into this type of plate and without the same degree of accuracy. The COPAS™ BIOSORT is capable of detecting and quantifying fluorescence in a population of nematodes but can only provide a cross sectional measure. However, by dispensing live worms into wells that contained the fluorescent dye SYTOX®, we are able to monitor the evolution of the fluorescent signal over time. This ability to measure fluorescence repeatedly in an individual worm was critical in facilitating longitudinal assays such as the heat shock assay we describe.

SYTOX® is a nucleic acid stain that has been used to assess cell viability. It cannot enter healthy cells, but will enter those cells whose cell membrane has been compromised, whereupon it binds to DNA and fluoresces. This dye is available in three preparations with different excitation and emission spectra (blue, green and orange). We first evaluated the utility of SYTOX® green as a score of nematode death in a manual thermotolerance assay by simply recording the time at which fluorescence occurred, assessed by visual inspection on a UV microscope, in a population of nematodes exposed to 35° C. heat shock. Using this score of death we observed the same survival kinetics as obtained using touch-provoked movement as the score of death (data not shown). This visual score of death was considerably faster to perform than assessing touch provoked movement, yet it remained subjective as a small amount of background fluorescence could be observed due to bacterial uptake. In addition, a significant amount of investigator time was still required to perform the score. We therefore evaluated the ability of the Fluoroskan Ascent fluorometric plate reader to detect and quantify the fluorescent signal to provide an objective and investigator-independent score. After testing a variety of microplate formats we found we were able to distinguish clearly between live and dead animals using low-volume black 384 well microtitre plates, sealed with an optically clear a proprietary sealant film, which prevented evaporation of the media within each well whilst maintaining optical clarity.

We validated the microplate assay by assessing its ability to detect thermotolerance differences between genetically altered strains of C. elegans and also in response to pharmacological intervention. Using the Fluoroskan software we were able to design a protocol such that the heat shock was performed in situ with measurements taken every 30 minutes over a 24 hour period. This ability to perform the heat shock incubation and simultaneously take measurements without removing the plate is a considerable improvement over the conventional assay and removes the need for any investigator input. A complete 384 well plate can be read in less than 30 seconds, with a 20 ms reading on each well, compared to the 20 minutes it would take to manually score the same number of worms fr touch provoked movement. Thus the automated reading is minimally 38.4 times faster than the manual score in a longitudinal assay, but more importantly is investigator independent.

Longitudinal assays have been used as a robust method of detecting differences between mutant strains of C. elegans when using touch provoked movement as a score (Lithgow et al. (1995)Proc. Natl. Acad. Sci., USA, 92: 7540-7544). One reason for using the longitudinal has been that the kinetics of a heat shock can be variable between one experiment and the next, making single time point cross sectional assays of limited use. For instance, although the difference in thermotolerance between N2 and age-1(hx546) is well-characterised and reproducible, the mean survival for each strain can vary between experiments. In particular the wild type strain is very susceptible to subtle environmental changes, such as periods of starvation, which can result in acquired thermotolerance. However, we have previously used single time point thermotolerance assays, termed “lock-in” assays to screen large numbers of worms from mutant screens (Sampayo et al. (2000) Ann N Y Acad Sci 908:324-326). By selecting those strains or lines that are thermotolerant in the “lock-in” assay for further analysis in a longitudinal assay, the process of large scale screening can be made much more efficient. The use of single time point assays, in which multiple plates are read once, combined with robotics and multiple plate readers, the throughput of this technique would increase to the point at which screening hundreds of thousands of worms per day would be possible.

Although we have demonstrated the use of this technique in the thermotolerance assay, in which survival in the face of an acute stress is measured, its application is much wider. Based on the correlation between increased stress resistance and lifespan we are interested in identifying compounds that confer thermotolerance, and in testing if they also extends lifespan. Similarly, we would examine compounds that protect against oxidative stress, induced by paraquat or hydrogen peroxide, and heavy metal toxicity. In each of the cases the endpoint is survival, scored by the absence of SYTOX®-associated fluorescence. It therefore follows that this assay could be applied to any paradigm in which survival is an endpoint, be it in response to a stressor or a nematocide for instance. We used SYTOX® green in our studies because its excitation and emission spectra were compatible with the filter sets installed on the Fluoroskan Ascent. However, other nucleic acid stains, such as SYTOX® blue, SYTOX® orange or propidium iodide could readily be used with the appropriate filters installed.

One of the major strengths of this high throughput method for detecting nematode fluorescence is that it can be applied to any paradigm in which nematode fluorescence, from any source, is the endpoint. Green fluorescent protein (GFP) has been used in nematode biology as a method of examining gene expression (Chalfie et al. (1994) Science 263: 802-805). Transgenic worms can readily be made by microinjection of a gene array, carrying the promoter of the gene of interest fused to the GFP gene, into the germline. The temporal regulation of this gene of interest can then be observed by the appearance of GFP-associated fluorescence. Generation of transgenic lines carrying GFP, or its related fluorophores YPP and CFP, downstream of a promoter could be used to screen for compounds that act by targeting specific genes causing an increase or decrease in fluorescence. We are investigating the ability of our system to detect a GFP signal by examining a transgenic line carrying the heat shock protein (hsp)-16 promoter upstream of GFP (Link et al. (1999) Cell Stress Chaperones 4: 235-242). Expression of GFP in this line is induced following heat shock and so this line will allow us to examine the time course of hsp-16 induction in response to heat stress in individual worms. Moreover it is possible to examine dual fluorescence, providing the opportunity to combine the kinetics of hsp-16::GFP induction with survival kinetics by examining death-associated fluorescence from SYTOX® orange, which fluoresces at a different wavelength.

The automated detection of fluorescence technique that we have described may not just be limited to the nematode Caenorhabditis elegans but to other model organisms as well. The COPAS™ technology from Union Biometrica has been developed for use with fruitfly (Drosophila melanogaster) embryos as well as zebrafish (Brachydanio rerio). Investigations in these species in which high throughput analysis of a fluorescent endpoint is required could also benefit from the applications we describe.

In summary we have developed a technique that allows for a rapid and objective score of survival in individual worms at rates many times faster than was previously possible, by the use of a fluorescent dye. By combining automated plate preparation and fluorometric quantitation we have further improved the throughput of this system also making it investigator independent. This system not only provides the opportunity to perform high throughput screens for compounds that affect nematode survival but can be applied to screens of pharmacological agents that affect any fluorescent signal of interest.

### Example 2

### An Automated High-Throughput Assay for Survival of the Nematode Caenorhabditis elegans

This example describes further studies of an automated high-throughput assay for survival of nematodes or other lower metazoans.

Although pharmacological intervention in the aging process has been demonstrated in C. elegans (Melov et al. (2000) Science, 289: 1567-1569; Harrington and Harley (1988) Mech. Ageing Dev. 43: 71-78) and Drosophila melanogaster (Kang et al. (2002) Proc. Natl. Acad. Sci. U.S.A., 99: 838-843) the number of compounds that have been investigated remains small. High-throughput screens for other compounds that elicit a similar effect in C. elegans have been limited by the necessity for microscopic inspection of individual worms to assess survival. Touch provoked movement is commonly used as the first indicator of death and remains the method of choice for most laboratories. This method works well in the hands of experienced investigators, yet the score remains somewhat subjective and is highly labor intensive. In order to develop a rapid and more objective measure of survival we have used a fluorescent dye, SYTOX® green, which fluoresces on DNA binding, yet will only enter cells whose membrane has been compromised (Roth et al. (1997) Appl. Environ. Microbiol., 63: 2421-2431). To facilitate an increase in throughput, we have employed COPAS™ BIOSORT automated worm handling technology to dispense individual nematodes into microtitre plates containing SYTOX dye, and used a fluorometric plate reader to quantify fluorescence.

Methods

Nematode Culture

Bristol N2 (wild-type), TJ1052[age-1(hx546) II], TJ1060[spe-9(hc88) I; fer-15(b26) II], TJ1062[spe-9(hc88) I; fer-15(b26) age-1(hx542)II] and E. coli OP50 were obtained from the Caenorhabditis Genetic Center at the University of Minnesota. Worms were maintained on 10 cm nematode growth medium (GM) agar plates carrying a lawn of E. coli OP50 or in liquid culture (S-medium (Sulston et al. (1988) Pp. 587-606 In Wood, W. B. eds. The nematode Caenorhabditis elegans. Cold Spring Harbor: Cold Spring Harbor Laboratory Press)) supplemented with E. coli OP50. Culture plates were maintained at 20° C., unless otherwise stated. Eggs were isolated by hypochlorite treatment of gravid adults (Id.).

Manual Thermotolerance Assay Using SYTOX Green Fluorescent Dye

Eggs were transferred to 24-well plates (Corning Inc., NY) containing 1 ml S-medium per well plus 1×10⁹ cells/ml E. coli OP50, and grown at 25° C. All assays were performed on 3 day old egg-laying hermaphrodites. To assess thermotolerance, SYTOX green (Molecular Probes Inc, OR) was added to each well (final concentration 1 μM) and the plate was incubated at 35° C. To score survival worms were examined under a fluorescence dissection microscope and worms exhibiting fluorescence were scored as dead.

Paraquat Resistance Assay Using SYTOX Green Fluorescent Dye

Eggs were transferred to 24-well plates (Corning Inc., NY) containing 1 ml S-medium, without cholesterol, plus 1×10⁸ cells/ml E. coli OP50, and grown at 25° C. All assays were performed on 3 day old egg-laying hermaphrodites. For worms treated with the superoxide/dismutase mimetic Euk-134 (Eukarion, Mass.) a single dose was added from a 5 mM stock (in Milli-Q water) on day one. At adulthood, 100 μl of culture media was removed from each well and replaced with 100 μl of paraquat stock solution (final concentration 40 mM). The cultures were then placed back at 25° C. and scored 48 h later for touch-provoked movement using a platinum wire. Immediately following the manual score, SYTOX green dye was added to each well to a final concentration of 1 μM. After 15 min the worms were examined under a fluorescence dissecting microscope and any animal exhibiting fluorescence was scored as dead.

Microplate Thermotolerance Assay

TJ1060[spe-9(hc88) I; fer-15(b26)II] and TJ1062[spe-9(hc88)I; fer-15(b26) age-1(hx542) II] hermaphrodites were grown at 15° C. to maintain fertile populations and allowed to develop at 25° C. to generate synchronous sterile populations. Assays were performed on 6 day old egg laying hermaphrodites grown at 15° C. or 3 day old sterile adults grown at 25° C. Worms (˜10,000 to 20,000) were washed off 10 cm NGM plates with 10 ml S-basal medium (Id.) into a 15 ml tube. After gravity settling for 5 min, the supernatant was aspirated and replaced with 9 ml S-basal medium. Following a further two washes the nematodes were introduced to the sample cup of a COPAS BIOSORT (Union Biometrica Inc., MA) at a concentration of approximately one worm per microliter. Worms were dispensed according to the manufacturers instructions, one animal per well, into 384-well black low-volume microtitre plates (Greiner Bio-One Inc, FL) containing 20 μl S-medium+1 μM SYTOX green with or without E. coli OP50, and the plate was sealed using VIEWseal plate sealing film (Greiner Bio-One Inc, FL) to prevent evaporation.

The thermotolerance assay was performed in a Fluoroskan Ascent fluorometer (Thermo Labsystems, MA). The temperature of the fluorometer was set to the indicated heat shock temperature and Ascent software was configured to measure the fluorescence from each well every 30 min over a 20-24 h period, with a 20 ms integration time for each well. At this setting a complete 384-well plate could be read within 30 s. For SYTOX green fluorescence the excitation wavelength was set to 485 nm and the emission wavelength 538 nm. Analysis of individual fluorescence curves to determine the time of death was performed using the Fluoroskan Ascent software. We found that a reliable score could not be obtained in 96 well plates, because the diameter of the detection beam relative to the well was such that a worm at the periphery of the well would not be detected. In contrast, using a 384-well plate the diameter of the beam and the well were identical, providing a more robust measurement.

Statistical Analysis

Differences in thermotolerance were assessed using the Mantel-Haenszel Logrank test as implemented in Prism® (GraphPad Software Inc.). Kaplan-Meier survival curves were generated by using Prism survival analysis.

Results

Manual Thermotolerance Assay Using SYTOX Green

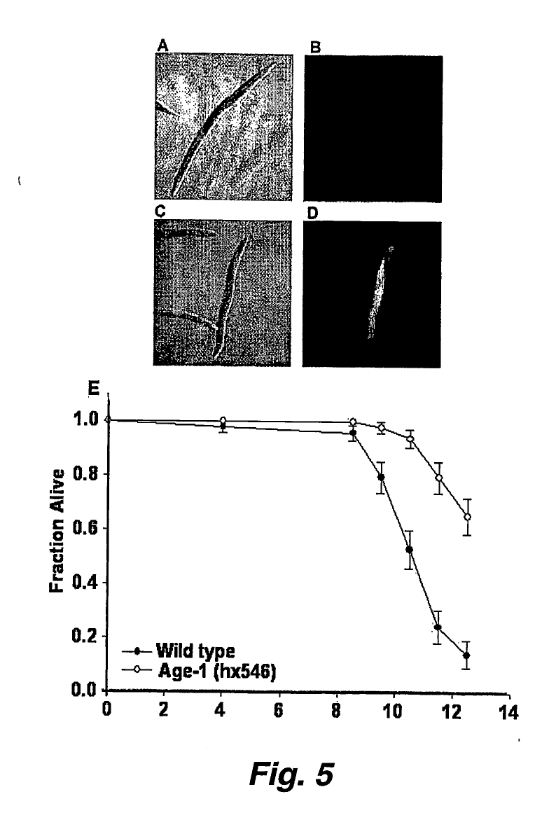

To evaluate the utility of SYTOX green dye in assessing survival, 3 day old gravid adult worms were exposed to a lethal thermal stress. Wild-type and worms carrying the age-1 mutation were grown in liquid culture in 24-well plates for 3 days at 25° C. (approximately 25 worms per well in duplicate). Prior to heat shock at 35° C. SYTOX green dye was added to a final concentration of 1 μM. Worms were examined by bright-field and fluorescence microscopy at 4 hr after the start of the heat shock and at intervals thereafter. There was no detectable fluorescence in live animals (FIG. 5, panels A and B). Fluorescence first appeared in the gonad and gut regions and over time became distributed throughout the body of the animal (FIG. 5, panels C and D). We scored death as the time at which fluorescence first appeared in the worm and the number of animals at each time point that exhibited fluorescence was used to construct a Kaplan-Meier survival curve (FIG. 5, panel E). Using the fluorescent score of death worms carrying the age-1 mutation were significantly more thermotolerant than wild-type (p<0.0001, Logrank test) as previously observed following a touch provoked movement protocol (Lithgow et al. (1995) Proc. Natl. Acad. Sci. U.S.A., 92: 7540-7544).

SYTOX Green Assay of Oxidative Stress Resistance

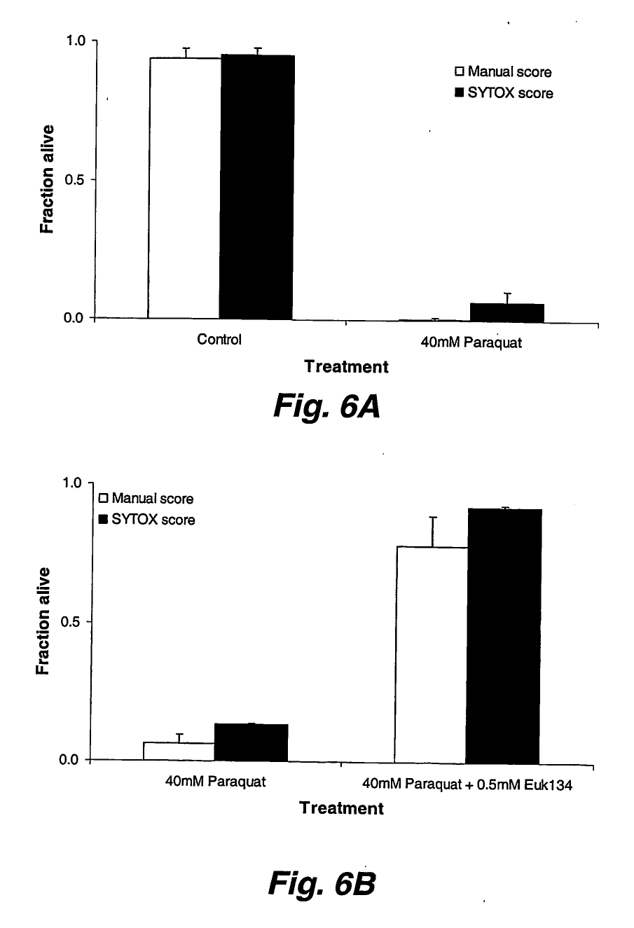

We then examined the utility of SYTOX green dye as a score of death due to oxidative stress by incubating 3 day old gravid adult worms in the presence or absence of 40 mM paraquat for 48 hr. Death was first scored by the absence of touch provoked movement and 15 min later by the presence of SYTOX-associated fluorescence (FIG. 6A). Using the manual score the mean fraction alive (±SEM) was 0.94±0.03 (n=69) in controls and 0.00±0.00 (n=157) with paraquat treatment. Following the addition of 1 μM SYTOX green dye and incubation for 15 min at room temperature, death was scored as the presence of fluorescence (FIG. 6A). The mean fraction alive (±SEM) as scored by SYTOX-associated fluorescence was 0.95±0.02 (n=69) in controls and 0.07±0.04 (n=157) in paraquat treated worms.

The utility of SYTOX dye was further tested in an experiment in which worms were cultured in the presence or absence of Euk-134 prior to paraquat exposure (FIG. 6B). Both the manual score and the SYTOX score identified the protective effect of 0.5 mM Euk-134 in response to paraquat treatment. The mean fraction alive (±SEM) scored by touch-provoked movement was 0.06±0.03 (n=47, 40 mM paraquat) and 0.78±0.11 (n=51, 40 mM paraquat+0.5 mM Euk134). The mean fraction alive (±SEM) scored using SYTOX dye was 0.13±0.00 (n=45, 40 mM paraquat) and 0.92±0.01 (n=51, 40 mM paraquat+0.5 mM Euk134). In each of these experiments the percentage of animals scored as alive using the SYTOX score was always slightly higher than the manual score.

Automated Thermotolerance Assay

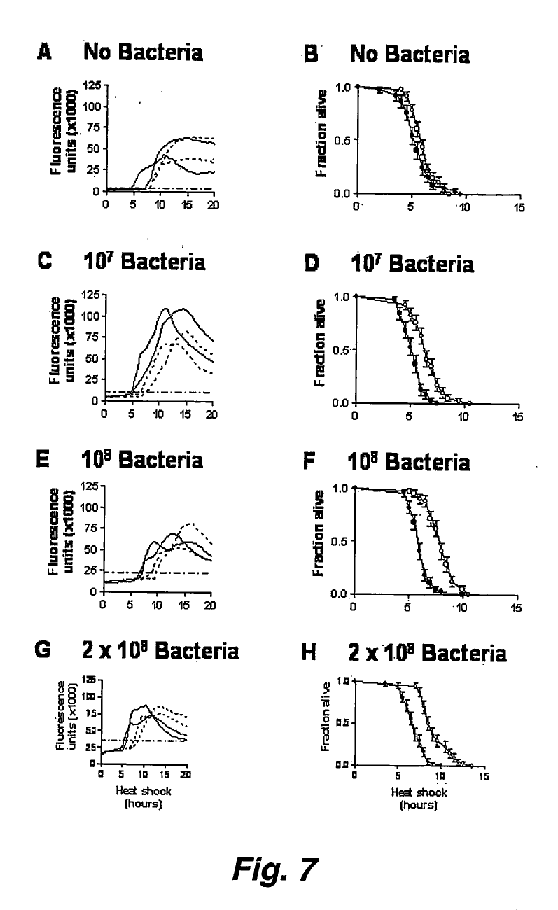

In order to increase the throughput and to generate a more objective score of survival we developed an automated assay of survival which used a plate-reading fluorometer to quantify the fluorescence of individual nematodes over time. Individual fluorescence curves were obtained for 6 day old egg laying hermaphrodite worms, grown at 15° C. and heat shocked at 37° C., and Kaplan-Meier survival curves were derived from the fluorescence data from one microplate assay (FIG. 7). Replication of these conditions in an additional two experiments yielded identical results. Worms were heat shocked in S-medium alone (FIG. 7, panels A and B), S-medium+10⁷ cells/ml E. coli OP50 (FIG. 7, panels C and D), S-medium+10⁸ cells/ml E. coli OP50 (FIG. 7, panels E and F) and S-medium+2×10⁸ cells/ml E. coli OP50 (FIG. 7, panels G and H). The initial baseline fluorescence reading increased with increasing concentrations of bacteria, and the presence of bacteria was associated with a gradual increase of fluorescence over time. At a concentration of 5×10⁸ cells/ml E. coli, and higher, it became difficult to discern the increase of fluorescence associated with a dead worm from the bacterial-derived fluorescence (data not shown). In FIG. 73 the time of death for an individual worm was calculated as the time at which the fluorescence exceeded the baseline (defined as the average of first 4 measurements) by an arbitrary threshold of 100%, using the fluorometer software. There was no significant difference in thermotolerance between wild-type and age-1 in S-medium alone. However, in the presence of E. coli OP50, age-1 was significantly more thermotolerant than wild-type (all p<0.0001, Logrank test) consistent with previous observations made by a variety of touch provoked movement assays (Lithgow et al. (1995) Proc. Natl. Acad. Sci. U.S.A., 92: 7540-7544; Lithgow et al. (1994) J. Gerontol., 49: B270-B276).

We further examined the effect of different thresholds of fluorescence in the determination of the point of death from the individual fluorescence curves (Table 1). In particular we wanted to ensure that the threshold level did not identify a time of death before the increase in fluorescence associated with nematode death. This issue became evident when using increasing concentrations of bacteria within the wells as the baseline fluorescence increased significantly over time in the absence of a worm. To provide a benchmark against which to compare the performance of the various cut-off criteria the individual fluorescence curves were examined to determine the time at which there was a significant increase in fluorescence over baseline (actual time of death). In S-medium without bacteria, the thermotolerance difference between wild-type and age-1(hx546) was small, but significant, using the actual time of death score but was not statistically significant at any of the other fluorescence thresholds. The magnitude of the difference in survival between the strains increased with increasing concentration of bacteria, as did the mean survival of each strain. A percentage increase over baseline fluorescence of 50% generated a large number of false positives (time of death scored earlier than the actual time of death). In contrast, a fluorescence threshold at or above 100% with at least 10⁸ cells/ml bacteria did not result in any false positive deaths. Increasing the threshold value did not affect the statistical difference between the two strains but acted to shift the survival curves to the right. The degree to which mean survival was right-shifted was identical for both strains indicating that the rate at which fluorescence accumulated following death was similar.

Table 1. Comparison of different cut-off criteria for determination of death events from individual nematode fluorescence curves. The actual time of death (no cut-off) was scored as the time at which fluorescence increased significantly over baseline and was determined by examination of individual fluorescence curves. The fluorometer software was used to automatically identify the time of death by computing the time at which fluorescence exceeded the baseline (calculated as the mean of the first 4 observations) by 50%, 100% and 200%. Mean survival ±SD at each cut-off for each condition was calculated and the Log-Rank test was used to assess statistical significance. False positives were identified as death events calculated by the fluorometer software that occurred before the actual time of death.

| | | Survival time (h) | | | | |
|---|---|---|---|---|---|---|
| | | TJ1060 | TJ1062 | | False positives | |
| | | mean ± SD | mean ± SD | | TJ1060 | TJ1062 |
| Condition | Cut-off | (n) | (n) | P-value | (n) | (n) |
| S-medium | None | 5.3 ± 0.9 | 5.9 ± 0.9 | <0.01 | — | — |
| | | (37) | (46) | | | |
| | 50% | 4.9 ± 1.4 | 5.6 ± 1.2 | n.s. | 20 | 27 |
| | | (37) | (46) | | | |
| | 100% | 5.5 ± 1.5 | 6.1 ± 1.0 | n.s. | 9 | 8 |
| | | (37) | (46) | | | |
| | 200% | 5.8 ± 1.7 | 6.4 ± 1.1 | n.s. | 2 | 3 |
| | | (37) | (46) | | | |
| S-medium + 10⁷ | None | 4.8 ± 1.1 | 6.2 ± 1.4 | <0.0001 | — | — |
| | | (38) | (41) | | | |
| cells/ml OP50 | 50% | 4.5 ± 1.4 | 6.1 ± 1.3 | <0.0001 | 13 | 16 |
| | | (38) | (41) | | | |
| | 100% | 5.3 ± 1.0 | 6.6 ± 1.4 | <0.0001 | 2 | 1 |
| | | (38) | (41) | | | |
| | 200% | 5.8 ± 1.2 | 7.1 ± 1.4 | <0.0001 | 0 | 0 |
| | | (38) | (41) | | | |
| S-medium + 10⁸ | None | 5.3 ± 1.0 | 7.1 ± 1.3 | <0.0001 | — | — |
| | | (44) | (42) | | | |
| cells/ml OP50 | 50% | 5.6 ± 1.0 | 7.2 ± 1.3 | <0.0001 | 3 | 14 |
| | | (44) | (42) | | | |
| | 100% | 6.1 ± 1.0 | 8.0 ± 1.3 | <0.0001 | 0 | 0 |
| | | (44) | (42) | | | |
| | 200% | 6.8 ± 1.2 | 8.9 ± 1.5 | <0.0001 | 0 | 0 |
| | | (44) | (42) | | | |
| S-medium + 2 × 10⁸ | None | 5.9 ± 1.2 | 8.2 ± 1.4 | <0.0001 | — | — |
| | | (43) | (40) | | | |
| cells/ml OP50 | 50% | 6.3 ± 1.2 | 8.4 ± 1.4 | <0.0001 | 2 | 6 |
| | | (43) | (40) | | | |
| | 100% | 7.0 ± 1.3 | 9.3 ± 1.6 | <0.0001 | 0 | 0 |
| | | (43) | (40) | | | |
| | 200% | 7.8 ± 1.4 | 10.8 ± 2.2 | <0.0001 | 0 | 0 |
| | | (43) | (40) | | | |

Discussion

To date more than 40 genes have been identified in C. elegans which when mutated lead to an increase in lifespan (Johnson et al. (2002) J. Inherit. Metab Dis. 25: 197-206), and all those tested have shown an increased resistance to acute stress (Lithgow and Walker (2002) Mech. Ageing Dev., 123: 765-771; Johnson et al. (2002) J. Inherit. Metab Dis. 25: 197-206). Moreover stress resistance has been shown to be a good surrogate measure of lifespan in screens for novel aging genes (Sampayo et al. (2000) Ann. N.Y. Acad. Sci., 908: 324-326; Munoz and Riddle (2003) Genetics 163: 171-180; Yang et al. (2000) Mech. Ageing Dev., 113: 101-116). Given the success of this approach a similar strategy could be applied to screening compounds for their affect on lifespan if high throughput methods with automated scoring were available. In this respect C. elegans has already been shown to be a promising bioassay for screening such compounds. The discovery that treatment of C. elegans with superoxide/catalase mimetics generated a significant increase in lifespan (Melov et al. (2000) Science, 289: 1567-1569) and oxidative stress resistance (J. Sampayo, A. Olsen and G. Lithgow, unpublished observation) demonstrated that pharmacological intervention in the aging process is possible. However, these experiments remain extremely labor intensive. Thus, the approach has been to target those compounds that are likely to impact on known aging pathways, rather than to screen thousands of compounds whose mechanism of action and molecular targets are not known. The development of the assay system we describe now makes this type of high-throughput screen a realistic proposition.

The development of this assay was achieved by the application of three separate technologies: the COPAS BIOSORT automated nematode sorting and dispensing platform, SYTOX green fluorescent dye and a microplate fluorometer. The COPAS BIOSORT “worm sorter” can sort and dispense nematodes on the basis of size, density and fluorescence. However, a limitation of this system is that it only generates cross-sectional information from a population of animals, and currently does not allow for repeated analysis of individual worms at the frequency we required. As we were interested in monitoring the time course of survival longitudinally in individual worms, we used the worm sorter to dispense single worms into the wells of a microtitre plate and measured fluorescence in a plate-reading fluorometer.

SYTOX green is a nucleic acid stain that has previously been used to assess cell viability (Roth et al. (1997) Appl. Environ. Microbiol., 63: 2421-2431). It cannot enter healthy cells, but will enter those cells whose cell membrane has been compromised, whereupon it binds to DNA and fluoresces upon excitation at 485 nm. We found that SYTOX green dye generated a strong fluorescent signal in dead nematodes, killed by incubation at 60° C. for 15 min, which could be detected easily under a fluorescence dissection microscope. This observation led us to evaluate the utility of SYTOX green as a score of nematode death in a manual thermotolerance assay by adding SYTOX dye to the culture medium and simply recording the time at which fluorescence occurred. Using this score of death we detected the same survival kinetics and thermotolerance difference between wild-type worms and those with the age-1 mutation previously observed using touch provoked movement as the score of death (Lithgow et al. (1995) Proc. Natl. Acad. Sci. U.S.A., 92: 7540-7544). We were further able to demonstrate the utility of the SYTOX dye in scoring death in response to the oxidative stressor, paraquat. We found SYTOX fluorescence to be an effective score of death not only in liquid culture but also on standard agar plates, where a single application of SYTOX solution to the surface of the plate was sufficient to score death in a longitudinal heat shock assay. This visual score of death was considerably faster to perform than assessing touch provoked movement, yet it still remains a somewhat subjective score. Fluorescence first appeared in the nematode as a small focal region in the gonad and gut regions and over time became distributed throughout the body of the animal. Thus, the investigator was required to make a judgement as to what degree of fluorescence constituted a death event.

The key step in developing a rapid, objective score was to employ a fluorometric plate reader to quantify the fluorescent signal generated by SYTOX. With an integration time per well of 20 ms it was possible to read fluorescence across the complete plate in less than 30 s, compared to the 20 min or more it would take to score the same number of worms for touch provoked movement (Table 2). In addition the Fluoroskan Ascent fluorometer was equipped with temperature control thus the heat shock could be performed in situ. The ability to generate fluorescence/survival curves for each individual nematode allowed a more objective assessment of the time of death to be made following completion of the assay.

TABLE 2
Comparison of manual and automated methods of assessing of
lifespan in C. elegans.

| | Manual | | Automated | |
|---|---|---|---|---|
| Number of worms | 200 | | 384 | |
| Time to score each timepoint | 20 | min* | 0.5 | min |
| Investigator time per hour | 20 | mm | 0 | |
| Length of Experiment | 18 | h | 18 | h |
| Total Investigator time | 6 | h | 0 | h |
| Worms scored per minute | 10 | | 768 | |
| Max number worms scored per hour | 600 | | 11520** | |

*Figure based on experienced investigator under ideal conditions
**Figure based on loading, reading and removing old plate and repeating with a new plate

We validated the microplate assay by assessing its ability to detect thermotolerance differences between genetically altered strains of C. elegans. We examined the wild-type and age-1 difference in a genetic background that confers sterility when grown at 25° C. (Fabian et al. (1995) Mech. Ageing Dev. 83: 155-170), as we thought that a sterile strain would remove the possibility of interference from progeny that may hatch and develop during the course of the assay. However, when sterile populations were evaluated we found that we could not reliably identify the time of death from individual fluorescence curves. Visual inspection of the microplate after the heat shock revealed that this was due to uptake of SYTOX by unfertilized oocytes laid within the well. Using fertile populations it transpired that interference from progeny was not an issue and thus all the data presented here were collected on fertile populations of TJ1060 and TJ1062 grown at 15° C. We also found that the concentration of bacteria within each well was an important factor in the microplate assay. A bacterial concentration within each well of at least 10⁸ cells/ml was required to maximize the thermotolerance difference between TJ1060 and TJ1062. However, at higher concentrations (above 5×10⁸ cells/ml) it was difficult to discern the point of nematode death due to bacterial uptake of SYTOX.

There are a number of modifications to this microplate method that would increase the throughput of the system still further. We have focused on a longitudinal heat shock assay which generates very detailed information on a relatively small population of animals. By using the “wormsorter” to dispense into multiple plates, reducing the number of times each plate is read and by performing the heat shock in an incubator rather than the fluorometer the throughput of this assay could be dramatically increased. In addition the measurement of fluorescence may be applied in other ways. We also evaluated the ability of our system to detect green fluorescent protein (GFP) driven by the heat shock protein-16 (hsp-16) transcriptional promoter (Link et al. (1999) Cell Stress. Chaperones. 4: 235-242) in live nematodes (data not shown). The ability to detect GFP suggests that this system could be used to identify compounds that act on specific gene promoters to enhance or inhibit gene expression by monitoring GFP-derived fluorescence. In addition it is possible, to combine the GFP signal with a fluorescent score of death using a dye that fluoresces at a different wavelength, such as SYTOX orange.

In summary, by combining the use of a fluorescent dye, automated plate preparation and fluorometric quantitation we have developed a technique that allows for a rapid and objective score of survival of individual worms, at rates many times faster than was previously possible. This system provides the opportunity to perform high-throughput screens for compounds that affect nematode survival in the face of acute stress and will facilitate the identification of drugs that extend nematode lifespan.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for screening for the activity of one or more agents on a nematode, said method comprising:
providing a microtiter plate comprising a plurality of wells, each of a plurality of said wells containing one or more nematodes;
contacting said nematodes with one or more test agents;
contacting said nematodes with a detectable label that indicates the viability of said nematodes; and
detecting said detectable label to ascertain the viability of nematodes contacted with said one or more test agents where an increase or decrease in the viability of the nematodes contacted with said test agents that the nematodes are susceptible to the activity of said test agent or that said test agent is active on said nematodes.

2. The method of claim 1, wherein said microtiter plate comprises at least 384 wells.

3. The method of claim 1, wherein said detectable label is a fluorescent label.

4. The method of claim 3, wherein said detectable label is a label that penetrates compromised cell membranes, but is substantially or completely excluded from live eukaryotic cells.

5. The method of claim 1, wherein said detectable label is selected from the group consisting of SYTOX® blue, SYTOX® orange, and propidium iodide.

6. The method of claim 4, wherein said label is SYTOX® cell stain.

7. The method of claim 3, wherein said detecting comprises detecting fluorescence in one or more wells comprising said microtiter plate with a fluorometer.

8. The method of claim 1, wherein said providing comprises dispensing one or more nematodes into a plurality of wells comprising a microtiter plate using an automated dispensing system.

9. The method of claim 8, wherein said providing comprises dispensing one or more nematodes into a plurality of wells comprising a microtiter plate using a COPAS™ BIOSORT device.

10. The method of claim 1, wherein said nematodes comprise Caenorhabditis sp.

11. The method of claim 1, wherein said nematodes comprise Caenorhabditis elegans.

12. The method of claim 11, wherein said nematodes comprise non-wildtype strains of C. elegans.

13. The method of claim 11, wherein said nematodes comprise genetic knock outs.

14. The method of claim 11, wherein said nematodes comprise transgenic nematodes.

15. The method of claim 11, wherein said nematodes comprise thermotolerant nematodes.

16. The method of claim 1, comprising subjecting said nematodes to heat and determining if said test agents increase or decrease thermotolerance of said nematodes.

17. The method of claim 1, comprising subjecting said nematodes to an agent that increases oxidative stress and determining if said test agents increase or decrease tolerance of oxidative stress by said nematodes.

18. The method of claim 1, comprising subjecting said nematodes to a nematocide and determining if said test agents increase or decrease tolerance of said nematocide by said nematodes.

19. The method of claim 1, wherein said method comprises providing a multiplicity of said microtiter plates.

20. The method of claim 1, wherein said detecting comprises reading a multiplicity of said microtiter plates.

21. The method of claim 1, wherein said microtiter plate is selected form the group consisting of a 96 well plate, a 100 well plate, a 320 well plate, a 384 well plate a 864 well plate, and a 1536 well plate.

22. The method of claim 21, wherein said microtiter plate is opaque.

23. A device for screening for the activity of one or more agents on a nematode, said device comprising:
a microtiter plate comprising a plurality of wells, each of a plurality of said wells containing one or more nematodes.

24. The device of claim 23, wherein the wells containing said one or more nematodes also contain a detectable label that indicates the viability of said nematodes.

25. The device of claim 24, wherein said detectable label is a fluorescent label.

26. The device of claim 25, wherein said detectable label is a label that penetrates compromised cell membranes, but is substantially or completely excluded from live eukaryotic cells.

27. The device of claim 24, wherein said detectable label is selected from the group consisting of SYTOX® blue, SYTOX® orange, and propidium iodide.

28. The device of claim 24, wherein said label is a SYTOX® cell stain.

29. The device of claim 23, wherein the wells containing said one or more nematodes also contain culture medium for said nematodes.

30. The device of claim 23, wherein said microtiter plate is selected from the group consisting of a 96 well plate, a 100 well plate, a 320 well plate, a 384 well plate a 864 well plate, and a 1536 well plate.

31. The device of claim 23, wherein said microtiter plate is opaque.

32. The device of claim 23, wherein at least 96 wells comprising said microtiter plate contain one or more nematodes.

33. The device of claim 23, wherein at least 384 wells comprising said microtiter plate contain one or more nematodes.

34. The device of claim 23, wherein at least 320 wells comprising said microtiter plate contain one or more nematodes.

35. The device of claim 23, wherein said microtiter plate is loaded with nematodes using an automated dispensing system.

36. The device of claim 35, wherein said automated dispensing system comprises a COPAS™ BIOSORT device.

37. The device of claim 23, wherein said nematodes comprise Caenorhabditis sp.

38. The device of claim 23, wherein said nematodes comprise Caenorhabditis elegans.

39. The device of claim 38, wherein said nematodes comprise non-wildtype strains of C. elegans.

40. The device of claim 38, wherein said nematodes comprise genetic knock outs.

41. The device of claim 38, wherein said nematodes comprise transgenic nematodes.

42. The device of claim 23, wherein said nematodes comprise thermotolerant nematodes.

43. A kit for screening for the activity of one or more agents on a nematode, said kit comprising:
a microtiter plate comprising a plurality of wells;
a plurality of nematodes; and
a detectable label that indicates the viability of said nematodes.

44. The kit of claim 43, wherein said plurality of nematodes are disposed such that each of a plurality of said wells contains one or more of said nematodes.

45. The kit of claim 43, wherein each of a plurality of said wells contains said detectable label.

46. The kit of claim 45, wherein a plurality of the wells containing nematodes also contain said detectable label.

47. The kit of claim 43, wherein said detectable label is a fluorescent label.

48. The kit of claim 43, wherein said detectable label is a label that penetrates compromised cell membranes, but is substantially or completely excluded from live eukaryotic cells.

49. The kit of claim 43, wherein said detectable label is selected from the group consisting of SYTOX® blue, SYTOX® orange, and propidium iodide.

50. The kit of claim 43, wherein said label is a SYTOX® cell stain.

51. The kit of claim 43, wherein the wells containing said one or more nematodes also contain culture medium for said nematodes.

52. The kit of claim 43, wherein said microtiter plate is selected from the group consisting of a 96 well plate, a 100 well plate, a 320 well plate, a 384 well plate a 864 well plate, and a 1536 well plate.

53. The kit of claim 43, wherein said microtiter plate is opaque.

54. The kit of claim 44, wherein at least 96 wells comprising said microtiter plate contain one or more nematodes.

55. The kit of claim 44, wherein at least 384 wells comprising said microtiter plate contain one or more nematodes.

56. The kit of claim 44, wherein at least 320 wells comprising said microtiter plate contain one or more nematodes.

57. The kit of claim 44, wherein said microtiter plate is loaded with nematodes using an automated dispensing system.

58. The kit of claim 57, wherein said automated dispensing system comprises a COPAS™ BIOSORT device.

59. The kit of claim 43, wherein said nematodes comprise Caenorhabditis sp.

60. The kit of claim 43, wherein said nematodes comprise Caenorhabditis elegans.

61. The kit of claim 60, wherein said nematodes comprise non-wildtype strains of C. elegans.

62. The kit of claim 60, wherein said nematodes comprise genetic knock outs.

63. The kit of claim 60, wherein said nematodes comprise transgenic nematodes.

64. The kit of claim 43, wherein said nematodes comprise thermotolerant nematodes.

65. The kit of claim 43, further comprising instructional materials teaching the use of said kit for high throughput screening for the effect of a test agent on a nematode.
